(19) United States
(12) Patent Application Publication
(10) Pub. No.: US 2021/0015644 A1
(43) Pub. Date: Jan. 21, 2021

(54) SYSTEM FOR DEPLOYING A VASCULAR BYPASS PROSTHESIS

(71) Applicant: CorQuest MedTech, Beauvechain (BE)

(72) Inventor: Bernard Segers, Bruxelles (BE)

(73) Assignee: CorQuest MedTech, Beauvechain (BE)

(21) Appl. No.: 16/976,031

(22) PCT Filed: Feb. 11, 2019
(86) PCT No.: PCT/EP2019/053259

§ 371 Date: Aug. 26, 2020

(30) Foreign Application Priority Data
Feb. 27, 2018 (BE) 2018/5116

(51) Int. Cl. A61F 2/958; A61F 2/97; A61F 2/954

(52) U.S. Cl. CPC A61F 2/958; A61F 2/97; A61M 2025/1013; A61F 2250/0067; A61F 2220/0016; A61F 2/954

(57) ABSTRACT

Systems are provided for deploying a vascular prosthesis having at least one stent with an end portion configured to be introduced into a vessel and a sheath connected thereto. The systems include a first expandable balloon for shaping and stabilizing the vascular prosthesis in the vessel, a second expandable balloon for sealing a junction between the end portion of the vascular prosthesis and the vessel, and means for expanding the first and second expandable balloons.

### FIELD OF THE INVENTION

The present invention relates to the field of vascular surgery, in particular that of arterial bypass surgery, for example between the aorta and the femoral arteries. The present invention more particularly relates to a system for deploying a vascular prosthesis for bypass surgery, comprising at least one stent.

The standard procedure for a bypass surgery between the aorta and the femoral arteries is open surgery. There therefore exists a need for a less invasive surgery. From the recent early stages of a laparoscopic aortobifemoral bypass surgery, this has been accepted by various authors as a less invasive alternative in the treatment of aortoiliac occlusive disease. Laparoscopy consists of an examination of the contents of the abdominal-pelvic cavity by means of an endoscope inserted through the abdominal wall.

### PRIOR ART

U.S. Pat. No. 8,357,190 describes a laparoscopic device for obtaining vascular access, in particular a deployment system for a laparoscopic conduit comprising an elongated graft tube having proximal and distal ends. The proximal end comprises at least two self-expanding stents for keeping the proximal end open, a gap being provided between the two stents to provide a sealed region through an opening in a wall of the vessel, and an elongated non-stented distal portion. The deployment system of U.S. Pat. No. 8,357,190 comprises technical construction requirements that makes it a complicated and expensive device. Furthermore, this laparoscopic device, while it enables minimally invasive surgery, has not yet been tested on humans. From its design, it could cause heavy bleeding during the deployment of the prosthesis. This is a major difficulty making this technique unpopular despite the obvious potential benefits for the patient. There therefore exists a need for a laparoscopic device of simpler construction and capable of making this surgery accessible to all vascular surgeons without causing bleeding.

US patent application 2003/135257 describes a system for deploying a vascular prosthesis for bypass surgery comprising two expandable balloons and means for expansion thereof.

Similarly, the system of US 2014/277361, EP 0 646 365 or WO 2016/022673 may comprise expandable balloons.

However, the information derivable from these publications still does not make this surgery accessible to all vascular surgeons without causing bleeding.

### SUMMARY OF THE INVENTION

This issue having been raised, the present invention is a solution to aorto-prosthetic anastomosis by a new construction of the system for deploying the vascular prosthesis. The term “anastomosis” refers here to the opening of a blood vessel in the direction of another, either directly or by connection (suture).

To that end, the present invention relates to a system for deploying a vascular prosthesis for bypass surgery comprising at least one stent, having an end portion to be introduced into a vessel and connected thereto, and comprising a sheath arranged to be introduced into the vessel and comprising a first expandable balloon for shaping and stabilising the prosthesis in the vessel and a second expandable balloon for sealing the junction between said end portion of the prosthesis and the vessel, the system further comprising means to expand (in other words, dilate) the balloons.

In the context of an arterial bypass surgery, for example between the aorta and the femoral arteries, the use of a vascular prosthesis comprising at least one stent and mounted on expandable balloons is essential, according to the present invention, in order to apply the section of the prosthesis equipped with a stent/stents inside the target artery and in order to seal the junction between the target artery and the extra-arterial vascular prosthesis.

In a preferred embodiment of the invention, the means for expanding the balloons comprise a first means for expanding the first expandable balloon and a second means for expanding the second expandable balloon.

Preferably, the first means and the second means of expansion are arranged so as to expand the balloons to different pressures and/or with different expansion kinetics. In this preferred embodiment, the balloons may be expanded independently of each other, both in time and from the point of view of the pressure (expansion/dilation factor). This allows, during the surgical procedure, the shape and stabilisation to be better adjusted, while effectively sealing the junction between the prosthesis and the aorta without risk of bleeding.

Preferably, the first expandable balloon is formed of a deformable biocompatible polymeric material, preferably an elastomer, for example a polyurethane, latex or silicone elastomer, and the second expandable balloon is formed of a non-deformable biocompatible polymeric material, for example a semi-crystalline polymer such as polyethylene, poly(ethylene terephthalate) or polyamide.

Preferably, the sheath is transparent and/or tearable in a lengthwise direction from its distal end.

In another preferred embodiment of the invention, the vascular prosthesis is bifurcated, in order to provide a strictly anatomical arterial bypass surgery. Preferably, a branch of the bifurcation is crossed by the assembly comprising the expandable balloons and the other branch remains free.

Finally, the proximal section of the vascular prosthesis may be equipped with means to grasp the inner wall of the blood vessel.

The deployment system of the present invention is a simpler construction than that of already cited U.S. Pat. No. 8,357,190, because it does not comprise means, such as a curved needle, enabling the puncture of the target vessel.

### DESCRIPTION OF A PREFERRED EMBODIMENT

The present invention will be better understood and illustrated by means of the attached drawings, which, however, only show one particular embodiment of the invention and consequently should not be interpreted as limiting the scope thereof, which is defined only by the claims.

In the attached drawings:

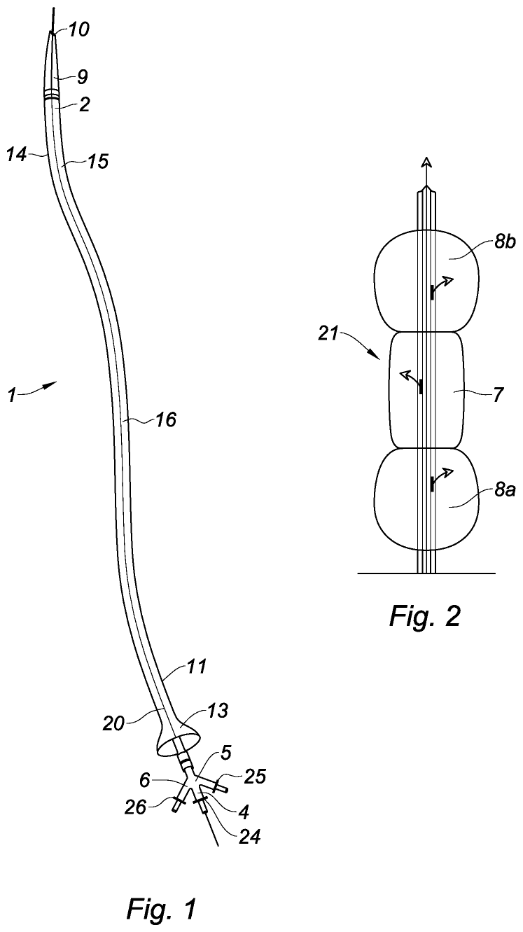

FIG. 1 shows a preferred embodiment of the system for deploying a vascular prosthesis of the invention;

FIG. 2 shows a system of expandable balloons capable of shaping and stabilising the vascular prosthesis inside and outside the vessel and sealing the junction between the prosthesis and the vessel;

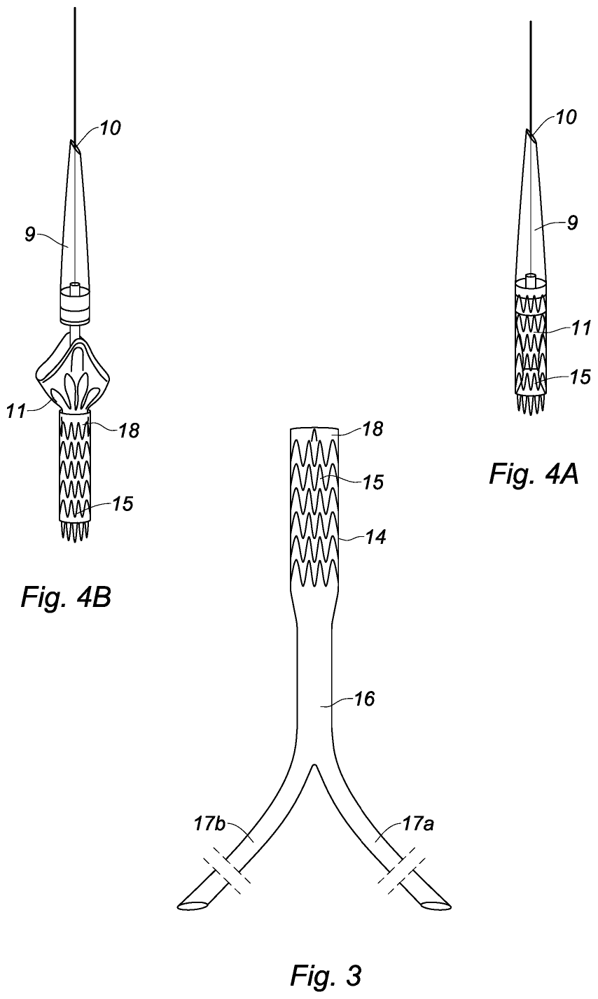

FIG. 3 shows an example of a bifurcated vascular prosthesis usable with the deployment system according to the invention and

FIGS. 4A and 4B show the inlet cone of the deployment system according to the invention at two stages of the surgical procedure, before and after deployment, respectively.

In the following description:
the terms “distal” and “proximal” should be understood conventionally in the art, i.e. as defined, for example, in U.S. Pat. No. 8,357,190,
the term “lumen”, referring to a blood vessel, indicates the internal space contained by its walls and
the term “stent” refers to any wire or tube, or any shaft, stick or rod, which can be inserted into the lumen of a blood vessel, particularly an artery.

Referring now to FIG. 1, a system for deploying 1 a vascular prosthesis 2 is described, in this case approximately 20 to 150 cm long according to the requirements of the proposed vascular surgery. The vascular prosthesis 2 may be in different forms and of different lengths and may particularly be bifurcated as shown in greater detail in FIG. 3. The vascular prosthesis 2 comprises on the one hand, at its proximal end 14, one or more stent(s) 15 (not shown in detail in FIG. 1) and on the other hand, at its distal end, a section which remains non-stented 16. The number of stents 15 depends on the patient and the proposed surgical procedure and can commonly reach 10. For example, the number of stents 15 may range from 2 to 10, or from 3 to 9, or from 4 to 8, or even from 5 to 7. The features of the stents 15, in particular their geometry, their size and the material from which they are formed are not crucial factors of the present invention and may be conventionally chosen in the art, in particular from commercially available models. The size of the stents 15, particularly their diameter, will be chosen based on the target blood vessel considered. Each stent 15 normally measures between 0.5 and 3 cm in width. As a non-limiting example, the distal diameter of the vascular prosthesis 2 may be in the order of 18 to 24 mm, and its distal end normally measures between 6 and 10 cm in length. The vascular prosthesis 2, with its different portions 14, 15 and 16, is designed to be introduced into a blood vessel such as an artery, particularly the aortic artery. For that reason, the vascular prosthesis 2 is preferably formed of polytetrafluoroethylene (abbreviated to PTFE, Teflon® brand), poly(ethylene terephthalate) (abbreviated to PET, Dacron® brand) or any other biocompatible polymeric material, in order to carry out an arterial bypass surgery. The different sections 14, 15, 16 of the vascular prosthesis 2 are included in a sheath 11 which is arranged to be introduced into the blood vessel. In a preferred embodiment, the sheath 11 is transparent in order to enable visualisation of the area equipped with stents 15 of the vascular prosthesis 2 and therefore to enable perfect positioning thereof in the artery. In another advantageous embodiment, the distal end 13 of the sheath 11 is tearable in a lengthwise direction, for example with the presence of a tear guide 20, in order to facilitate its extraction when the vascular prosthesis 2 has been deployed, as shown in FIG. 1.

The distal end of the deployment system comprises an assembly 21 (FIG. 2) comprising, in this case, an expandable balloon 8a for opening the prosthesis when it is positioned in the aorta and designed to perfect the deployment of the distal section 16 (FIG. 3), another expandable sealing balloon 7 for sealing the junction between the end portion 14 of the prosthesis and the vessel and particularly an expandable balloon 8b for shaping the prosthesis designed to open it when it is in the aorta and to press it against the aorta and ensure its stability. This balloon 8b has been described above as a first balloon; the balloon 7, as a second balloon, is therefore here positioned between balloons 8a and 8b. The proximal end of the deployment system 1 is covered by an inlet cone 9 whose end 10 is bevel-shaped, or any other form appropriate to facilitate its introduction into the blood vessel without damaging it. The distal end 13 of the sheath 11 has a flared shape to enable or facilitate the withdrawal of the system after deploying the vascular prosthesis 2. At this end, the deployment system 1 comprises the means 5, 6, 25, 26 for expanding the balloons of the balloon assembly 21. At that same end, the deployment system 1 may also comprise a means 4 for injecting the physiological serum, or any other liquid product used in endovascular surgery, into the axial lumen to ensure advantageous lubrication.

In the assembly 21, the expandable balloon 7 is therefore arranged between the two expandable balloons 8a and 8b. The number and dimensions of the balloons are not requirements of the present invention and could be changed, taking the particular proposed surgical procedure into account. Even though, in certain cases, a greater number of balloons could in theory improve the efficiency of the system, it should be appreciated that, in general, the number of balloons does not need to be greater than 3.

In the embodiment described here, the expandable balloons 8a and 8b are formed of a deformable biocompatible polymeric material. The possibility of deforming this polymeric material is such that the first balloon 8b enables the prosthesis (including the stent(s) 15) to be opened while moulding the diameter of the vessel (for example, the aorta) without oversizing it, so as to avoid too great a pressure on the walls of the vessel (artery). As for the balloon 8a, it ensures the deployment of the non-stented distal section 16 of the prosthesis outside the vessel. Numerous examples of deformable biocompatible polymeric materials are known to one skilled in the art and are commercially available, such as biocompatible elastomers. In an embodiment of the invention, said deformable biocompatible polymeric material comprises a polyurethane elastomer, combined with another elastomer where applicable, or is formed mainly of polyurethane. In another embodiment, said deformable biocompatible polymeric material may be a latex or silicone elastomer.

The second expandable sealing balloon 7 is here formed of a mainly non-deformable biocompatible polymeric material. The non-deformability of this polymeric material is such that this second balloon 7 enables the section equipped with a stent/stents 15 of the vascular prosthesis 2 to be applied inside the target vessel (artery) in a stable manner, and to effectively seal the junction area between the end of the prosthesis and the vessel. Numerous examples of non-deformable biocompatible polymeric materials are known to one skilled in the art and are commercially available, such as biocompatible semi-crystalline polymers. Semi-crystalline materials have, contrary to amorphous materials, a highly ordered molecular structure with high melting points. They do not gradually soften according to an increase in temperature; on the contrary, they remain solid until a given amount of heat has been absorbed. In one embodiment of the invention, examples of such semi-crystalline polymers comprise polyamide, polyethylene, polypropylene, ethylene-propylene copolymers and biocompatible polyesters such as polyethylene terephthalate and poly-hydroxyalkanoates.

Referring now to FIG. 1 again, the balloon expansion system is now described in more detail. The means for expanding the balloons comprise pressure supply sources, such as conduits 5, 6, having shut-off valves or through valves 25, 26 to dilate, through the conduits 5, 6, the balloons according to different pressures and/or according to different expansion kinetics. In this way, it is possible to face the difficulties which could appear to the vascular surgeon watching the deployment procedure on screen, such as too great a pressure on the artery and/or too great a pressure on the prosthesis, while regulating the expansion factor applied to each section. In a preferred embodiment of the invention, the balloon 7 has a predetermined diameter for sealing the junction of the prosthesis and the vessel. As a non-limiting example, a pressure of 6 atmospheres is generally suitable for a diameter of 5 mm and a pressure of 8 atmospheres for a diameter of 5.1 mm. FIG. 1 here shows a central conduit 4 for, the valve 24 being open, injecting the physiological serum, or any other liquid product used in endovascular surgery, into the axial lumen.

Referring to FIG. 3, an example of a bifurcated vascular prosthesis 2 adapted to the deployment system 1 of the invention is now described. In this preferred embodiment of the invention, the vascular prosthesis is bifurcated at the distal level of its non-stented section 16 in order to be able to propose a strictly anatomical bypass surgery. For example, it bifurcs by over 6 to 8 cm into two different stems (branches) 17a, 17b, each measuring between 15 and 40 cm in length and between 7 and 10 mm in diameter. The stem 17b, which is not connected to the section equipped with stents 15, is preferably closed at its distal end, so as to avoid leaks during the deployment. It is also possible to grasp this stem with a surgical clip. FIG. 3 also shows, at the other proximal end of the vascular prosthesis 2, grasping means 18, here surgical hooks or staples, for grasping the inner wall of the vessel.

Referring to FIGS. 4a and 4b, it is shown in more detail how the proximal end portion of the system 1 comprising at least one stent 15, located at the proximal end of the sheath 11, is equipped with a cone 9. Preferably, as shown in these figures, the end 10 of the cone 9 is bevelled in order to facilitate the introduction into the artery of the patient without damaging the wall of the vessel. As a non-limiting example, the distal end of the cone 9 has an external diameter of 0.8 mm and gradually reaches, over 2 to 5 cm, the diameter of the sheath 11.

All the dimensions (length, diameter, etc.) of the different components of the deployment system according to the present invention are only mentioned here as an indication corresponding to the most common cases, but should not be interpreted as limiting parameters of the invention.

1. A system for deploying a vascular prosthesis for bypass surgery, the vascular prosthesis comprising a stent having an end portion configured to be introduced into a vessel and a sheath connected thereto and arranged to be introduced into the vessel, the system comprising:
a first expandable balloon configured for shaping and stabilizing the vascular prosthesis in the vessel;
a second expandable balloon configured for sealing a junction between the end portion of the vascular prosthesis and the vessel; and
means for expanding the first expandable balloon and the second expandable balloon,
wherein the first expandable balloon is formed of a deformable biocompatible polymeric material and the second expandable balloon is formed of a non-deformable biocompatible polymeric material.

2. The system for deploying a vascular prosthesis according to claim 1,
wherein the vascular prosthesis comprises a distal section,
wherein the system further comprises a third expandable balloon arranged to open the distal section of the vascular prosthesis when it is positioned in the vessel.

3. The system according to claim 1, further comprising a conduit for injecting a physiological lubrication serum therein.

4. The system according to claim 1, wherein the sheath comprises, at a distal end thereof, a tear guide.

5. The system according to claim 1, wherein the means for expanding the first expandable balloon and the second expandable balloon comprise first means for expanding the first balloon and second means for expanding the second balloon.

6. The system according to claim 5, wherein the first means and the second means are differently arranged to at least one of expand the first and second balloons to different pressures or expand the first and second balloons with different expansion kinetics.

7. The system according to claim 1, wherein the vascular prosthesis is bifurcated at a distal end thereof.

8. The system according to claim 7, wherein the vascular prosthesis comprises a first branch crossed by the first and second balloons, and a second branch which is not crossed by the first and second balloons.

9. The system according to claim 1, wherein the vascular prosthesis is equipped with means for grasping an inner wall of the vessel.

10. The system according to claim 2, further comprising a conduit for injecting a physiological lubrication serum therein.

11. The system according to claim 10, wherein the sheath comprises a tear guide at a distal end thereof.

12. The system according to claim 11, wherein the means for expanding the first expandable balloon and the second expandable balloon comprise first means for expanding the first balloon and second means for expanding the second balloon.

13. The system according to claim 12, wherein the first means and the second means are differently configured so as to at least one of expand the first and second balloons to different pressures or expand the first and second balloons with different expansion kinetics.

14. The system according to claim 13, wherein the vascular prosthesis is bifurcated at the distal end thereof.

15. The system according to claim 14, wherein the vascular prosthesis comprises a first branch crossed by first and second balloons, and a second branch which is not crossed by the first and second balloons.

16. The system according to claim 15, wherein the vascular prosthesis is equipped with means for grasping an inner wall of the vessel.

17. A system for deploying a vascular prosthesis for bypass surgery, the vascular prosthesis comprising a stent having an end portion configured to be introduced into a vessel and a sheath connected thereto and arranged to be introduced into the vessel, the system comprising:
a first expandable balloon configured for shaping and stabilizing the vascular prosthesis in the vessel;
a second expandable balloon configured for sealing a junction between the end portion of the vascular prosthesis and the vessel; and
at least one valved conduit for expanding the first expandable balloon and the second expandable balloon,
wherein the first expandable balloon is formed of a deformable biocompatible polymeric material and the second expandable balloon is formed of a non-deformable biocompatible polymeric material.

18. The system according to claim 17, wherein the at least one valved conduit comprises a first valved conduit for expanding the first balloon and a second valved conduit for expanding the second balloon.

19. The system according to claim 17, wherein the first valved conduit and the second valved conduit are arranged to at least one of expand the first and second balloons to different pressures or expand the first and second balloons with different expansion kinetics.

20. The system according to claim 17, wherein the vascular prosthesis is equipped with hooks or staples configured for grasping an inner wall of the vessel.
